(19) United States
(12) Patent Application Publication
(10) Pub. No.: US 2026/0151565 A1
(43) Pub. Date: Jun. 4, 2026

(54) ADJUSTING MEDICAMENT DELIVERY PARAMETERS IN AN OPEN LOOP MEDICAMENT DELIVERY MODE

(71) Applicant: INSULET CORPORATION, Acton, MA (US)

(72) Inventors: Joon Bok LEE, Acton, MA (US); Jason O'CONNOR, Acton, MA (US); Yibin ZHENG, Hartland, WI (US); Jonathan HARDY, Reading, MA (US)

(21) Appl. No.: 19/460,852

(22) Filed: Jan. 27, 2026

Related U.S. Application Data
(63) Continuation of application No. 18/119,062, filed on Mar. 8, 2023, now Pat. No. 12,564,679.
(60) Provisional application No. 63/318,209, filed on Mar. 9, 2022.

(51) Int. Cl. A61M 5/172; G16H 20/17

(52) U.S. Cl. CPC A61M 5/1723; G16H 20/17; A61M 2230/201

(57) ABSTRACT

The exemplary embodiments provide an automated approach for adjusting the medicament delivery rate to the user when operating in an open loop manner (“open mode”). The approach relies upon an insulin delivery history to the user make adjustments to the medicament delivery rate in the open mode. In particular, the exemplary embodiments may look at the medicament delivery history while the medicament delivery device is operating in a closed loop manner (“closed mode”) to determine how to adjust the open mode medicament delivery rate. It is presumed that in closed mode, the control system of the medicament device has gained knowledge over time about how to control the medicament delivery rate to produce good treatment outcomes for the user. The exemplary embodiments leverage this knowledge to adjust the open mode medicament delivery rates. Medicament bolus deliveries in open mode may also be adjusted in like fashion.

### RELATED APPLICATIONS

This application is a continuation of U.S. patent application Ser. No. 18/119,062, filed Mar. 8, 2023, which claims the benefit of U.S. Provisional Patent Application No. 63/318,209, filed Mar. 9, 2022, the contents of which are incorporated herein by reference in their entirety.

### BACKGROUND

Conventional medicament delivery devices may operate in a closed loop manner and/or an open loop manner. In the closed loop manner, a feedback loop is provided to adjust a delivery rate to achieve a target objective. For example, where the medicament delivery device is an insulin delivery device, the objective may be to obtain a target glucose level for a user. The difference between a current glucose level and the target glucose level may be fed back to adjust the medicament delivery rate. When a medicament delivery device operates in the open loop manner, the user provides inputs to the medicament delivery device that determine the dosage of delivery of the medicament delivery. For example, the user may specify the medicament delivery amounts explicitly in inputs. The inputs may be used to create a profile that is stored and used by the medicament delivery device when operating in the open loop manner.

One of the drawbacks for operating a medicament delivery device in an open loop manner is that users tend to select medicament delivery dosages that are sub-optimal. For instance, with insulin delivery devices operating in an open loop manner, users tend to underestimate their insulin needs and choose too low of medicament delivery dosage. As a result, users have less than optimal glucose control, and users tend to deliver insulin boluses more as a result. The increased delivery of insulin boluses increases the risk of the user suffering from hypoglycemia.

### SUMMARY

In accordance with a first inventive aspect, a non-transitory storage medium stores instructions executable by a processor of an electronic device having a display to cause the processor to, for a medicament delivery device that operates in an open loop mode and a closed loop mode, determine a first average, which is an average over a period of a total basal delivery amount of medicament that is delivered to a user by the medicament delivery device in the closed loop mode during a same segment of time in each interval the period. The interval may be a day, multiple days, hours, etc. The instructions also cause the processor to compare the first average with an amount of medicament that is programmed to be delivered by the medicament delivery device to the user for the segment of time in the open loop mode to determine a difference. The instructions additionally cause the processor to, based on the comparing, perform the following: display on the display a suggestion of a change in the amount of medicament that is programmed to be delivered to the user by the medicament delivery device for the segment of time in the open loop mode to reduce the difference, and/or programmatically cause the amount of medicament that is programmed to be delivered to the user by the medicament delivery device for the segment of time in the open loop mode to be adjusted to reduce the difference.

The medicament may be one of insulin, glucagon like peptide-1 (GLP-1), pramlintide, a co-formulation of any of the foregoing, or another agent affecting glucose level. The programmatically causing the amount of medicament that is programmed to be delivered to the user by the medicament delivery device for the segment of time in the open loop mode to be adjusted may include calculating a second average of the first average with the amount of the medicament that is programmed to be delivered by the medicament delivery device to the user for the segment of time in the open loop mode, and adjusting the amount of the medicament that is programmed to be delivered to the user by the medicament delivery device for the segment of time in the open loop mode based on the second average to produce an adjusted amount of the medicament that is programmed to be delivered to the user by the medicament delivery device for the segment of time in the open loop mode. The adjusting of the amount that is programmed to be delivered to the user by the medicament delivery device for the segment of time in the open loop mode based on the second average to produce an adjusted amount may include adding a weighted version of the amount of the medicament that is programmed to be delivered to the user by the medicament delivery device for the segment of time in the open loop mode with a weighted version of the second average to produce an adjusted amount of the medicament that is programmed to be delivered to the user by the medicament delivery device for the segment of time in the open loop mode to produce the adjusted amount of the medicament.

A magnitude of the adjusting of the amount of the medicament that is programmed to be delivered to the user by the medicament delivery device for the segment of time in the open loop mode to reduce the difference may depend on a magnitude of the difference or a magnitude of the suggested adjusting in the suggestion of the amount of the medicament that is programmed to be delivered to the user by the medicament delivery device for the segment of time in the open loop mode to reduce the difference may depend on the magnitude of the difference. The non-transitory storage medium may further store instructions that cause the processor to: define a plurality of ranges of possible values for the difference, define a change amount for each range, determine a selected one of the ranges that the difference falls into, and wherein either the suggestion of the change in the amount of the medicament suggests the change amount of the selected one of the ranges or the programmatically causing the amount of the medicament that is programmed to be delivered to the user by the medicament delivery device for the segment of time in the open loop mode is adjusted by the change amount of the selected one of the ranges. If the programmatically causing the amount of the medicament that is programmed to be delivered to the user by the medicament delivery device for the segment of time in the open loop mode to be adjusted to reduce the difference is performed, the non-transitory storage medium further may store instructions that cause the processor to display a magnitude of the adjusting of the amount of the medicament that is programmed to be delivered to the user by the medicament delivery device for the segment of time in the open loop mode on the display.

In accordance with another inventive aspect, an electronic device is provided that includes a non-transitory storage media for storing computer programming instructions and a processor. The processor is configured to execute the computer programming instructions to cause the processor to, for a medicament delivery device that operates in an open loop mode and a closed loop mode, determine a first average which is an average over a period of a total basal delivery amount of medicament that is delivered to a user by the medicament delivery device in the closed loop mode during a same segment of time in intervals in the period. The instructions also cause the processor to compare the first average with an amount of medicament that is programmed to be delivered by the medicament delivery device to the user for the segment of time in the open loop mode to determine a difference. The instructions further cause the processor to, based on the comparing, perform the following: display on the display a suggestion of a change in the amount of medicament that is programmed to be delivered to the user by the medicament delivery device for the segment of time in the open loop mode to reduce the difference, and/or programmatically cause the amount of medicament that is programmed to be delivered to the user by the medicament delivery device for the segment of time in the open loop mode to be adjusted to reduce the difference.

The electronic device may be one of the medicament delivery device or a management device for the medicament delivery device. The medicament may be one of insulin, glucagon like peptide-1 (GLP-1), pramlintide or another agent affecting glucose level. The processor configured to programmatically causing the amount of medicament that is programmed to be delivered to the user by the medicament delivery device for the segment of time in the open loop mode to be adjusted may comprise the processor being configured to: calculate a second average which is an average of the first average with the amount of the medicament that is programmed to be delivered by the medicament delivery device to the user for the segment of time in the open loop mode, and adjust the amount of the medicament that is programmed to be delivered to the user by the medicament delivery device for the segment of time in the open loop mode based on the second average to produce an adjusted amount of the medicament that is programmed to be delivered to the user by the medicament delivery device for the segment of time in the open loop mode. A magnitude of the adjusting of the amount of the medicament that is programmed to be delivered to the user by the medicament delivery device for the segment of time in the open loop mode to reduce the difference may depend on a magnitude of the difference or a magnitude of the suggested adjusting in the suggestion of the amount of the medicament that is programmed to be delivered to the user by the medicament delivery device for the segment of time in the open loop mode to reduce the difference may depend on the magnitude of the difference.

The processor may be further configured to execute additional programming instructions to: define a plurality of ranges of possible values for the difference, define a change amount for each range and determine a selected one of the ranges that the difference falls into; and either the suggestion of the change in the amount of the medicament may suggest the change amount of the selected one of the ranges or the programmatically causing the amount of the medicament that is programmed to be delivered to the user by the medicament delivery device for the segment of time in the open loop mode may be adjusted by the change amount of the selected one of the ranges. If the programmatically causing the amount of the medicament that is programmed to be delivered to the user by the medicament delivery device for the segment of time in the open loop mode to be adjusted to reduce the difference is performed, the processor may further be configured to execute programming instructions to display a magnitude of the adjusting of the amount of the medicament that is programmed to be delivered to the user by the medicament delivery device for the segment of time in the open loop mode on the display.

In accordance with an additional inventive aspect non-transitory storage medium storing instructions executable by a processor of an electronic device having a display to cause the processor to, for a medicament delivery device that operates in an open loop mode and a closed loop mode, determine an average over a period of a total basal delivery amount of medicament that is delivered to a user by the medicament delivery device in the closed loop mode during a same segment of time in intervals in the period and compare the average with an amount of medicament that is programmed to be delivered by the medicament delivery device to the user for the segment of time in the open loop mode to determine a difference. The instructions further cause the processor to, based on the determined difference, adjust at least one of the insulin to carbohydrate ratio (ICR) of the user or the correction factor of the user and use the adjusted ICR of the user or the adjusted correction factor of the user in determining a recommended bolus dosage of medicament for the user.

Both the ICR of the user and the correction factor of the user may be adjusted in the adjusting. The non-transitory storage medium may store programming instructions to cause the processor to, based on the comparing, perform the following: display on the display a suggestion of a change in the amount of medicament that is programmed to be delivered to the user by the medicament delivery device for the segment of time in the open loop mode to reduce the difference, and/or programmatically cause the amount of medicament that is programmed to be delivered to the user by the medicament delivery device for the segment of time in the open loop mode to be adjusted to reduce the difference. The programming instructions may cause the processor to perform both the displaying on the display of the suggestion and the programmatically causing the amount of medicament that is programmed to be delivered to the user by the medicament delivery device for the segment of time in the open loop mode to be adjusted to reduce the difference. The medicament may be one of insulin, glucagon like peptide-1 (GLP-1), pramlintide or another agent affecting glucose level. The processor may be part of a management device for the medicament delivery device.

### BRIEF DESCRIPTION OF THE DRAWINGS

FIG. 1 depicts an illustrative medicament delivery system suitable for exemplary embodiments.

FIG. 2 depicts a flowchart of illustrative steps that may be performed by exemplary embodiments in selecting whether to operate in an open loop mode or a closed loop mode.

FIG. 3 depicts a flowchart of illustrative steps that may be performed in exemplary embodiments to create a basal profile.

FIG. 4A depicts an illustrative user interface of an exemplary embodiment that may be used in generating a basal profile.

FIG. 4B depicts a graphical depiction of an illustrative basal profile.

FIG. 5 depicts a flowchart of illustrative steps that may be performed in exemplary embodiments to perform open loop basal delivery.

FIG. 6A depicts a flowchart of illustrative steps that may be performed in exemplary embodiments to adjust delivery rates for open loop basal delivery.

FIG. 6B depicts an illustrative user interface for displaying a suggested adjustment in an open loop basal delivery rate.

FIG. 7 depicts a flowchart of illustrative steps that may be performed in exemplary embodiments to determine a recommended adjustment to a basal delivery rate in accordance with a first option.

FIG. 8 depicts a flowchart of illustrative steps that may be performed in exemplary embodiments to determine a recommended adjustment to a basal delivery rate in accordance with a second option.

FIG. 9 depicts a flowchart of illustrative steps that may be performed in exemplary embodiments to adjust bolus delivery dosages.

### DETAILED DESCRIPTION

The exemplary embodiments provide an automated approach for adjusting the medicament delivery amounts to the user when operating in a manual or an open loop manner (“open loop mode”). The approach relies upon an insulin delivery history to the user make adjustments to the medicament delivery amounts in the open loop mode. In particular, the exemplary embodiments may look at the medicament delivery history while the medicament delivery device is operating in a closed loop manner (“closed loop mode”) to determine how to adjust the open loop mode medicament delivery amounts. In closed loop mode, the control system of the medicament device gains knowledge over time about how to control the medicament delivery rate to produce more optimal treatment outcomes for the user. The exemplary embodiments leverage this knowledge to adjust the open loop mode medicament delivery amounts. These amounts may be expressed as hourly delivery rates in some embodiments, but other units may be used.

The adjustments may help the performance of the treatment provided by the medicament delivery device. For example, for an insulin delivery device, the adjustments may result in better glucose control for the user while the insulin delivery device is operating in open loop mode. The adjustments may also reduce the number of boluses needed by the user and hence, reduce the risk of hypoglycemia to the user.

In some exemplary embodiments, the medicament delivery amounts that are adjusted are those for basal medicament deliveries in open loop mode (e.g., according to a user's basal profile) that occur at regular intervals each cycle (where a cycle may be, for instance, a 5-minute interval) on an ongoing basis, though the rate may be in units of medicament units per hour. However, the exemplary embodiments may also alter medicament bolus dosages as well in a proportional manner. For the case where the medicament is insulin, the control system may adjust the insulin to carbohydrate ratio (ICR) for the user and the correction factor (CF) for the user in an amount that is proportional to the adjustment amount to the basal medicament delivery rate in open loop mode. Thus, if the basal medicament delivery rate is adjusted up by 10%, the ICR and CF for the user are adjusted up by 10%.

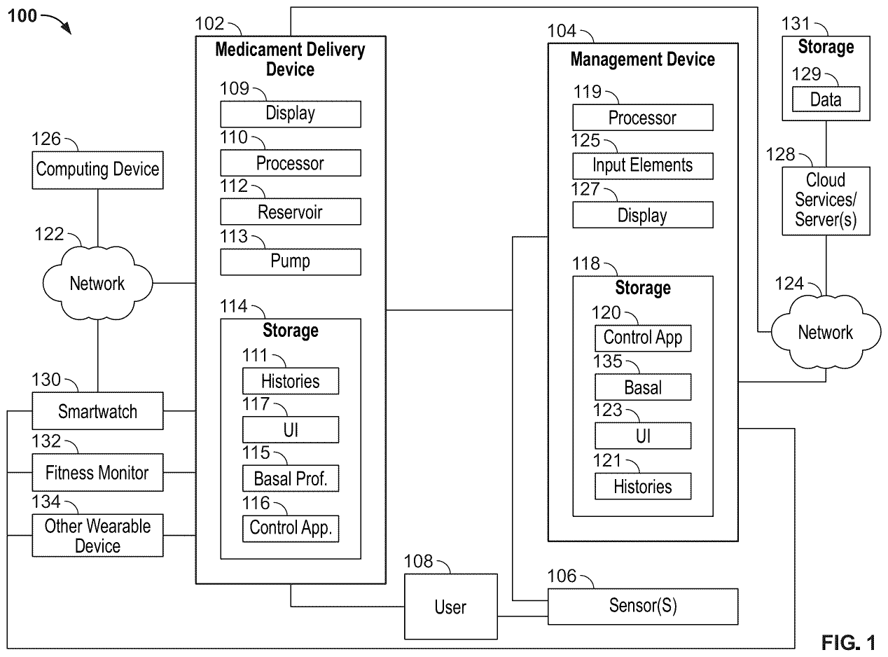

FIG. 1 depicts an illustrative medicament delivery system 100 that is suitable for delivering a medicament to a user 108 in accordance with the exemplary embodiments. The medicament delivery system 100 includes a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) or carried by the user (e.g., on a belt or in a pocket) with the medicament delivery device 102 being connected to an infusion site where the medicament is injected using a needle and/or cannula. In a preferred embodiment, a surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface 117 that may include one or more display screens shown on display 109. The display 109 may display information to the user 108 and, in some instances, may receive input from the user 108, such as when the display 109 is a touchscreen.

The control application 116 may control delivery of a medicament to the user 108 per a control approach like that described herein. The storage 114 may hold histories 111 for a user, such as a history of basal deliveries, a history of bolus deliveries, and/or other histories, such as a meal event history, exercise event history, glucose level history and/or the like. The storage 114 also may include one or more basal profiles 115 that are used when the medicament delivery device is operating in open loop mode. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include one or more housings for housing its various components including a pump 113, a power source (not shown), and a reservoir 112 for storing a medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site.

There may be one or more communications links with one or more devices physically separated from the medicament delivery device 102 including, for example, a management device 104 of the user and/or a caregiver of the user, a sensor 106, a smartwatch 130, a fitness monitor 132 and/or another variety of wearable device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof.

The medicament delivery system 100 may or may not also include a management device 104. In some embodiments, no management device is not needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensors 106. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. The one or more control applications 120 may be responsible for controlling the medicament delivery device 102, such as by controlling the AID delivery of insulin to the user 108. The storage 118 may store the one or more control applications 120, histories 121 like those described above for the medicament delivery device 102, one or more basal profiles 135 and other data and/or programs.

A display 127, such as a touchscreen, may be provided for displaying information. The display 127 may display user interface (UI) 123. The display 127 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128. The cloud services/server(s) 128 may provide output from the model 115 as needed to the management device 104 and/or medicament delivery device 102 during operation.

Other devices, like smartwatch 130, fitness monitor 132 and wearable device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage or to request, initiate, or confirm delivery of a bolus of a medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data.

A wide variety of medicaments may be delivered by the medicament delivery device 102. The medicament may be insulin for treating diabetes. The medicament may be glucagon for raising a user's glucose level. The medicament may also be a glucagon-like peptide (GLP)-1 receptor agonists for lowering glucose or slowing gastric emptying, thereby delaying spikes in glucose after a meal. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agents, a nutritional supplement or a vitamin.

The functionality described below for the exemplary embodiments may be under the control of or performed by the control application 116 of the medicament delivery device 102 or the control application 120 of the management device 104. In some embodiments, the functionality may be under the control of or performed by the cloud services or servers 128, the computing device 126 or by the other enumerated devices, including smartwatch 130, fitness monitor 132 or another wearable device 134.

The medicament delivery device 102 may operate in an open loop mode and in a closed loop mode. In the open loop mode, the user 108 manually inputs the amount of medicament to be delivered (such as per hour) for segments of the day. The inputs may be stored in a basal profile 115, 135 for the user 108. In other embodiments, a basal profile may not be used. The control application 116, 120 uses the input information from the basal profile 115, 135 to control basal medicament deliveries in open loop mode. In contrast, in the closed loop mode, the control application 116, 120 determines the medicant delivery amount for the user 108 on an ongoing basis based on a feedback loop. For an insulin delivery device, the aim of the closed loop mode is to have the user's glucose level at a target glucose level.

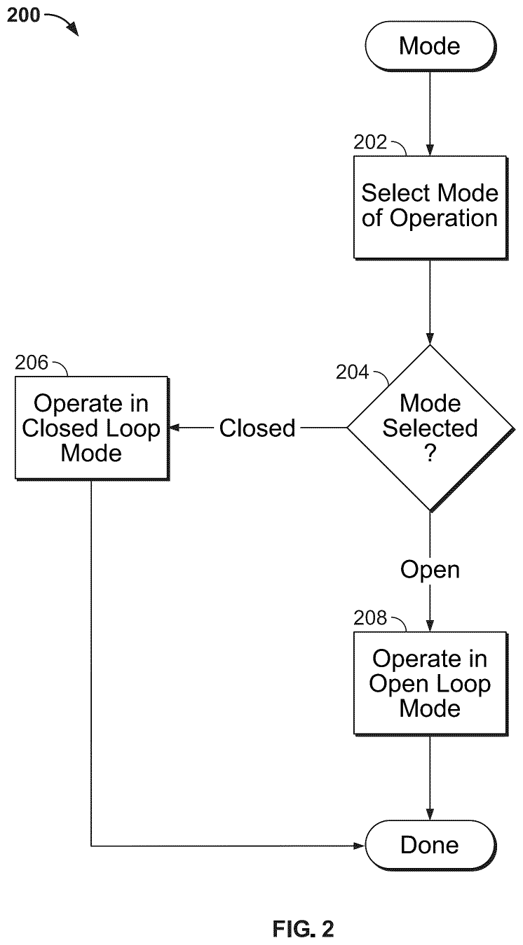

FIG. 2 depicts a flowchart 200 of illustrative steps that may be used in selecting a mode in some exemplary embodiments. At 202, user and/or the control application 116, 120 may select the mode. For instance, the user interface 117, 123 may include a user interface element, like a button or menu item, that allows the user 108 to change the current mode. The medicament delivery device 102 may have a default mode, such as the closed loop mode. At 204, based upon the selection, the control application 116, 120 determines the proper mode. At 206, if the closed loop mode is selected, the medicament delivery device operates in the closed loop mode. At 208, if the open loop mode is selected, the medicament delivery device 102 operates in the closed loop mode.

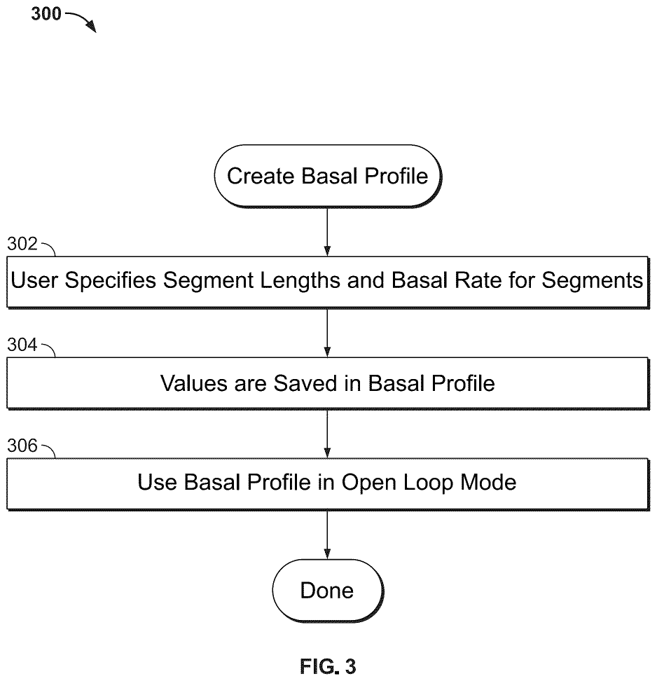

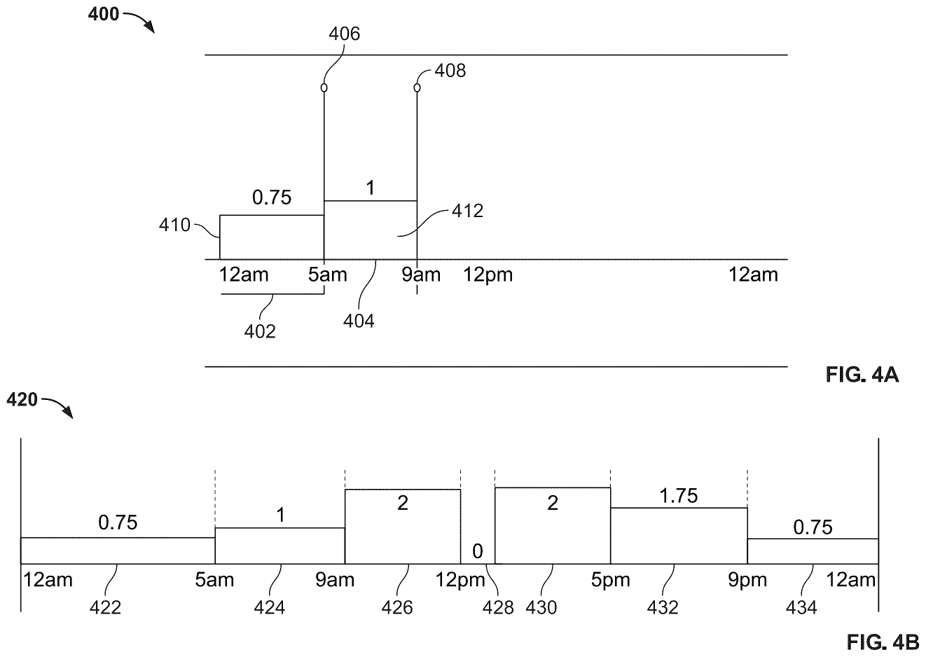

As mentioned above, in some exemplary embodiments, open loop mode uses a basal profile 115, 135 to guide medicament deliveries in open loop mode. FIG. 3 depicts a flowchart 300 of illustrative steps that may be performed to create a basal profile 115, 135 for the user 108. It should be appreciated that the user 108 may have multiple basal profiles 115, 135 and may select among them when operating in open loop mode. The user interface 117, 123 may provide options for the user to create a basal profile 115, 135. At 302, the user specifies segment lengths and basal medicament delivery rates for the segments. Each segment is a period of time, whose length is specified by the user 108. FIG. 4A depicts an example user interface 400 where a user 108 has begun to create a basal profile. In the depicted example, the user 108 has defined medicament delivery rates for segment 402 and segment 404. The first segment 402 begins at 12 am and continues to 5 am, where the second segment 404 runs from 5 am to 9 am. The user 108 may specify the length of each segment, such as by dragging bars 406 and 408 along a timeline to specify the start time and end time for the segment. The user 108 also enters the delivery rate over the specified segment time frame and the user may do this by entering numerical numbers on a numerical user interface or by adjusting the height of a bar (for example) on the user interface by dragging a top of the bar up or down with the user's finger. By way of example, the medicament delivery rate for segment 402 is 0.75 units per hour as indicated by the height of the graphic bar 410, whereas the medicament delivery rate for segment 404 is 1.0 unit per hour as indicated by the height of graphic bar 412. This process is repeated for remaining segments of a day to cover all 24 hours of the day. FIG. 4B shows an illustrative completed profile 420 having segments 422, 424, 426, 428, 430, 432 and 434.

At 304, the values for the segment length and the associated hourly medicament delivery rates for the segments that have been specified by the user are stored in a basal profile 115, 135. At 306, the basal profile is used by the control application 116, 120 in open loop mode. Thus, suppose that the basal profile 420 of FIG. 4B is used for a user in manual mode or open loop mode. In that case, the control application 116, 120 sets the basal medicament delivery rate at 0.75 units between 12 am and 5 am. At 5 am, the control application 116, 120 boosts the delivery rate to 1.0 unit per hour until 9 am.

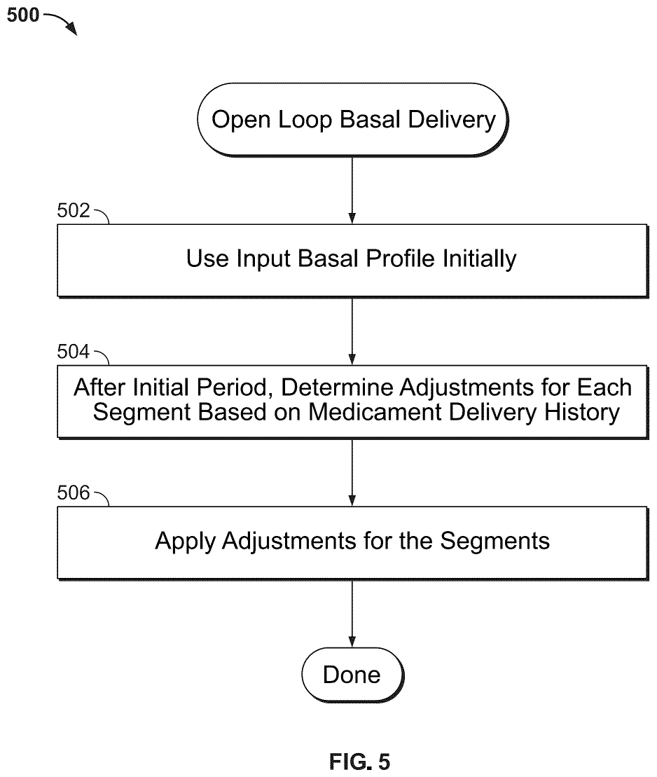

As mentioned above, the exemplary embodiments may adjust the user-input hourly medicament delivery rates. In some embodiments, the adjusted values may be stored in a corrected basal profile stored with the basal profiles 115, 135, and corrected basal profile may be used by the control application 116, 120 when operating in open loop mode. FIG. 5 depicts a flowchart 500 of illustrative steps that may be performed in exemplary embodiments to make adjustments to the medicament delivery amounts provided by the user 108. At 502, the user enters the values for incorporation in the basal profile 115, 135 to set the medicament delivery amounts of the medicament delivery device. Subsequently, at 504, adjustments to the medicament delivery amounts used in open loop mode may be made based on the medicament delivery history in closed loop mode. For example, after 48 hours of operation in closed loop mode, there is sufficient medicament delivery history to rely upon for making adjustments. Hence, adjustments can be made after that time of operation. At 506, the adjustments are made for the medicament delivery amounts (e.g., delivery amounts per hour) specified by the basal profile 115, 135. These adjustments may be made to the basal profile 115, 135 or to the actual value that is set by the control application 116, 120 for the medicament delivery amounts. The adjustment may be triggered by a transition from closed loop mode to open loop mode, may be triggered at regular intervals, such as every day, every 12 hours, or another period, or may be triggered by specific events, such as hypoglycemic and/or hyperglycemic events that may occur throughout the day or week.

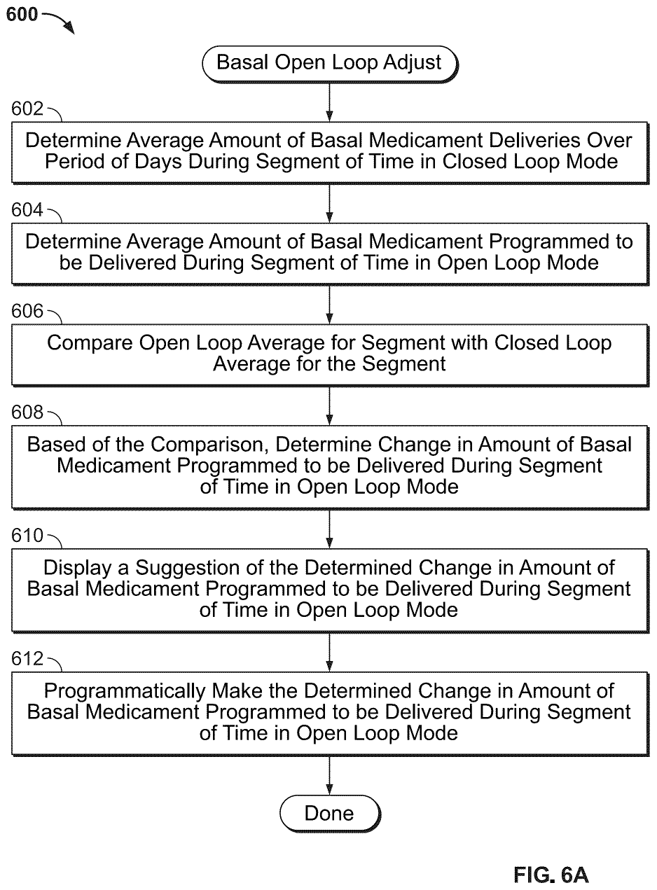

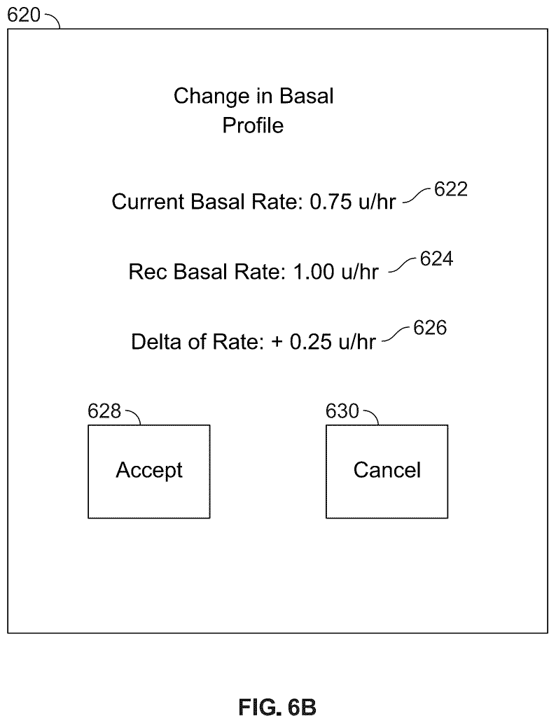

FIG. 6A depicts a flowchart 600 of illustrative steps that may be performed in exemplary embodiments to determine and to make adjustments to the medicament delivery amounts specified in the basal profile 115, 135. The aim of making the adjustments is to improve the performance of the medicament delivery device 102 in open loop mode by choosing medicament delivery rates that are better suited to the user 108. The exemplary embodiments rely upon medicament delivery rate history in the closed loop mode to determine what an appropriate medicament delivery amount is and to calculate the magnitude of adjustments that need to be made. At 602, the average amount of basal medicament deliveries over a period of intervals, such as days, during a segment of time in closed loop mode is calculated. For an insulin delivery device, this average TOT_{AID}(j) may be calculated as:

$${TOT}_{AID} ( j ) = \frac{{\sum }_{k = 1}^{m} {M}_{AID} ( j )}{m}$$

$$where$$

$${M}_{AID} ( j ) = {\sum }_{i = {t}_{start} , j}^{{t}_{end , j}} {I}_{k} ( i )$$

and where m is the number of days in the period over which Iₖ(i) is summed, and Iₖ(i) is the amount of basal insulin delivered to the user 108 during cycle i in segment j, whether by manual delivery or by automated insulin delivery from an algorithm.

At 604, the amount of basal medicament to be delivered during the segment of time to be delivered during the segment of time in open loop mode is determined. For the segment j, the total amount of medicament delivered during the segment is determined. This step entails looking up the delivery rate for the medicament in open loop mode in the basal profile. Where the medicament is insulin, the amount of basal metabolic medicament in open loop may be expressed as:

$${TOT}_{manual} ( j ) = {h}_{j} \cdot {M}_{man} ( j )$$

where hⱼ is the number of hours in the segment j and Mₘₐₙ(j) is the hourly insulin delivery rate for the segment j.

At 606, the determined open loop amount is compared with the closed loop average for the segment. At 608, based on the comparison of the determined open loop amount with the closed loop average, a change amount for the basal medicament delivery rate during the segment in open loop mode is determined, as will be detailed below. In general, the magnitude of the change amount is reflective of the magnitude of the difference between the open loop amount and the closed loop average for the segment.

At 610, a suggestion of the determined change amount in the basal medicament that is programmed to be delivered during the segment of time in open loop mode may be generated and displayed on a display such as display 109 or display 127. FIG. 6B shows an example of an illustrative display screen 620. The display screen 620 lists the current basal medicament delivery rate 622. In this illustrative case, the current basal medicament delivery hourly rate is 0.75 units/hour. The display screen 620 also shows the recommended basal medicament hourly delivery rate 624. In this illustrative case, the recommended basal medicament delivery rate is derived from the average amount of basal medicament deliveries during the segment and is expressed as an hourly rate of 1.0 units per hour. The difference or a delta between the current basal medicament delivery rate in the recommended basal medicament delivery hourly rate 626 may also be displayed on the display screen 620. The user 108 is thus informed of the proposed change in delivery amounts and can select the accept button 628 to accept the change or the cancel button 630 to reject the change.

At 612, the change in the amount of basal medicament programmed to be delivered during the segment of time in open loop mode is programmatically made. The change may be made in response to the user selecting the accept button 628 or may be automatically made in some embodiments. It should be appreciated that in some exemplary embodiments no suggestion is displayed; rather the change is performed automatically without the user having to approve the change.

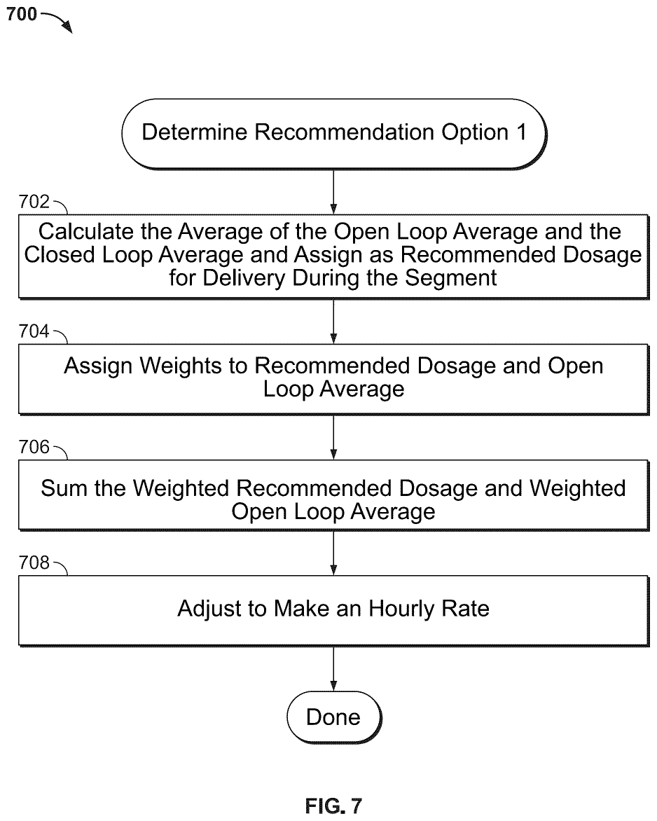

There are different options for determining the magnitude of the change that should be made to the basal medicament delivery amount in the open loop mode. FIG. 7 depicts a flowchart 700 of steps that may be performed in exemplary embodiments in accordance with a first option. At 702, the open loop mode amount currently specified for the current segment and the closed loop mode average are summed and divided by two to produce a recommended medicament delivery amount for the segment. The idea behind this operation is to use the average of the closed loop mode average for the segment and the open loop mode amount for the segment as the recommended amount TOT_{rec}(j). This operation can be expressed as:

$${TOT}_{rec} ( j ) = \frac{{TOT}_{AID} ( j ) + {TOT}_{manual} ( j )}{2}$$

In other examples, this recommended amount may completely favor the automated insulin delivery amount TOT_{AID}(j), or an asymmetric weighing of the total manual insulin delivery amount and the automated insulin delivery amount, such as the following equation:

$${TOT}_{rec} ( j ) = A \cdot {TOT}_{AID} ( j ) + ( 1 - A ) \cdot {TOT}_{manual} ( j )$$

where A is a value between 0 and 1.

At 704, weights are then assigned to the closed loop mode average amount for the segment and to the recommended amount, and at 706 the weighted values are summed. An equation for these operations is:

$${TOT}_{manual , new} ( j ) = B \cdot {TOT}_{manual} ( j ) + ( 1 - B ) {TOT}_{rec} ( j ) .$$

where B is the weighing of previous manual basal segment recommendation versus the current recommended amount. A value of 0.8 for B brings about a more gradual movement towards TOT_{rec}(j) so that change is not too rapid and there is not an overshoot. Lastly, at 708, the amount TOT_{manual,new}(j) is divided by the number of hours in the segment to produce an hourly rate. This can be expressed as:

$${b}_{rec} ( j ) = \frac{{TOT}_{manual , new} ( j )}{{h}_{j}} .$$

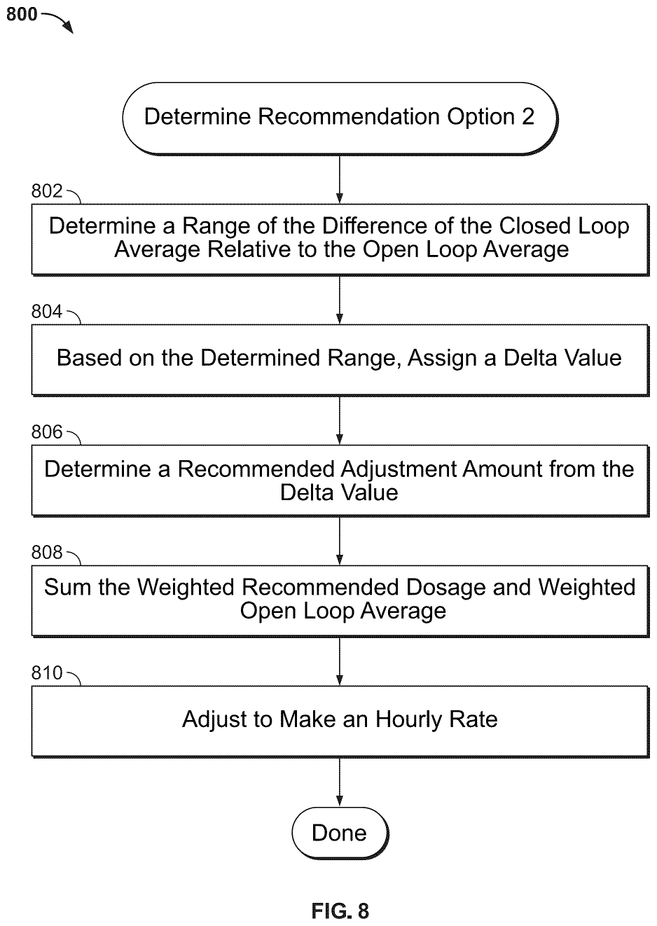

FIG. 8 depicts a flowchart 800 of illustrative steps that may be performed in exemplary embodiments to determine a changed amount for medicament delivery during a segment j. At 802, the difference between the closed loop average and the open loop amount is determined, and it is determined what range of differences the determined difference falls into. At 804, based on the determined range for the difference, a delta value is assigned. An illustrative formulation for steps 802 and 804, where the medicament is insulin, is:

$${TOT}_{delta} ( j ) = \{ \begin{matrix}- 0.1 & {TOT}_{AID} ( j ) \leq 0.8 {TOT}_{manual} ( j ) \\ - 0.05 & 0.8 {TOT}_{manual} ( j ) < {TOT}_{AID} ( j ) \leq 0.95 {TOT}_{manual} ( j ) \\ 0 & 0.95 {TOT}_{manual} ( j ) < {TOT}_{AID} ( j ) \leq 1.05 {TOT}_{manual} ( j ) \\ 0.05 & 1.05 {TOT}_{manual} ( j ) < {I}_{AID} ( j ) \leq 1.2 {TOT}_{manual} ( j ) \\ 0.1 & 1.2 {TOT}_{manual} ( j ) < {TOT}_{AID} ( j )\end{matrix}$$

In this example, categories for which TOT_{delta} are placed are in increments of 0.1 around 100% of TOT_{AID}, but the width of these categories can be varied, such as in increments of 0.75, and can even be made asymmetric to reduce the risk of overcorrection and overdelivery based on higher AID delivery, such as limiting the maximum TOT_{delta} adjustment category to 1.2TOTₘₐₙᵤₐₗ(j)<TOT_{AID}(j). Further, in this example, the TOT_{delta} is incremented in units of 0.05 per categorical change—however, these units can vary in quantities, such as 0.1, 0.15, or others.

At 806, the recommended dosage amount is determined from the delta value. One suitable equation is:

$${TOT}_{rec} ( j ) = {TOT}_{manual} ( j ) ( 1 + {TOT}_{delta} ( j ) ) .$$

At 808, the recommended dosage is weighted, and the open loop mode amount is weighted. The weighted values are summed, such as:

$${TOT}_{manual , new} ( j ) = B \cdot {TOT}_{manual} ( j ) + ( 1 - B ) {TOT}_{rec} ( j ) .$$

At 810, TOT_{manual,new}(j) is converted to an hourly rate by dividing it by the number of hours in the segment j:

$${b}_{rec} ( j ) = \frac{{TOT}_{manual , new} ( j )}{{h}_{j}} .$$

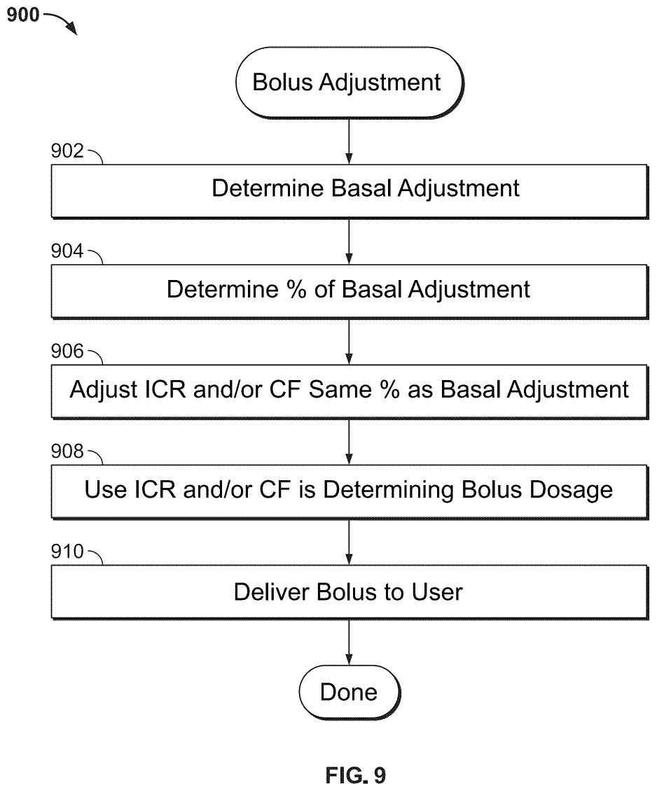

The exemplary embodiments may also change parameters that affect bolus medicament deliveries. FIG. 9 depicts a flowchart 900 of illustrative steps that may be performed to make such adjustments in exemplary embodiments. At 902, the basal medicament delivery rate adjustment for open loop mode is determined as described above relative to FIGS. 6A-8. At 904, the control application 116, 120 determines what percentage of change has occurred in the basal medicament delivery rate for open loop mode as a result of the adjustment. For example, if the basal medicament amount was adjusted from 0.75 units per hour to 1.00 units per hour for a segment, the percentage of change would be +33% (i.e., +0.25/0.75). At 906, the ICR and/or the CF for the user 108 may be adjusted the same percentage as the basal medicament delivery rate (e.g., +33%). The change in the ICR and/or CF for the user 108 will result in a change in bolus amount for the user 108. For example, if the user 108 wishes to compensate for a meal containing 20 grams of carbohydrates and a ICR of 1 unit of insulin to 10 grams of carbohydrates is normally used, a 33% adjustment to the ICR means that 2.66 units of insulin rather than 2.00 units of insulin would be delivered in the bolus after the adjustment. At 908, the adjusted ICR and/or CF are used to determine the medicament bolus dosage, and at 910, the medicament bolus is delivered to the user 108.

In the present application, it should be understood that the term “non-transitory storage medium” may either be a single non-transitory storage medium or multiple non-transitory storage mediums which may be contained in a single device or may be contained (or split up) in different devices, e.g. one non-transitory storage medium in a delivery device and another one in a management device. Similarly, the term “processor” may be a single processor or multiple processors which may be contained in a single device or may be contained (or split up) in different devices, e.g. one processor in a delivery device and another one in a management device.

While exemplary embodiments have been described herein, it should be appreciated that various changes in form and detail may be made without departing from the intended scope as defined in the appended claims.

1. A method for configuring an open-loop medicament delivery profile specifying programmed medicament delivery amounts for a plurality of time segments for a user, comprising:
performing automated closed-loop medicament delivery to the user in a closed loop mode of operation over a calibration period, during which medicament delivery amounts are determined by a control algorithm based on physiological feedback;
aggregating, for each of the plurality of time segments, a total amount of medicament delivered during the calibration period while operating in the closed-loop mode;
detecting a transition request from the closed-loop mode to an open-loop mode of operation;
deriving, in response to the transition request, an open-loop delivery profile in which programmed medicament delivery amounts for the plurality of time segments are adjusted as a function of the aggregated closed-loop delivery amounts; and
operating the medicament delivery device in the open-loop mode using the adjusted open-loop delivery profile.

2. The method of claim 1, wherein the calibration period comprises a plurality of full daily cycles of closed-loop operation.

3. The method of claim 1, wherein aggregating the total amount of medicament delivered comprises summing basal medicament delivery amounts occurring at regular control intervals within each time segment across the calibration period.

4. The method of claim 1, wherein deriving the open-loop delivery profile comprises weighting the aggregated closed-loop delivery amounts with previously programmed open-loop delivery amounts to limit the magnitude of change.

5. The method of claim 1, wherein deriving the open-loop delivery profile comprises limiting adjustments to a maximum percentage change per calibration event.

6. The method of claim 1, wherein deriving the open-loop delivery profile uses asymmetric weighting such that upward adjustments and downward adjustments are bounded by different limits.

7. The method of claim 1, wherein the derivation is automatically triggered by detection of a transition from closed-loop mode to open-loop mode.

8. The method of claim 1, wherein the derivation is suppressed unless the calibration period exceeds a minimum time threshold.

9. A non-transitory storage medium storing instructions executable by a processor to cause a medicament delivery system to:
partition a recurring daily cycle for a user into a plurality of time segments defined independently of physiological measurements;
during a closed-loop mode of operation, determine a reference medicament delivery model comprising, for each time segment, a representative medicament delivery amount derived from actual medicament delivery commanded by a closed-loop control algorithm across multiple occurrences of the time segment;
store the reference medicament delivery model separately from a user-entered open-loop delivery profile; and
generate an updated open-loop delivery profile by modifying the user-entered open-loop delivery profile toward the reference medicament delivery model for one or more of the time segments.

10. The non-transitory storage medium of claim 9, wherein the representative medicament delivery amount for each time segment is an average basal delivery amount across multiple days.

11. The non-transitory storage medium of claim 9, wherein the representative medicament delivery amount excludes bolus medicament deliveries.

12. The non-transitory storage medium of claim 9, wherein modifying the user-entered open-loop delivery profile comprises blending the user-entered profile with the reference medicament delivery model using a configurable weighting factor.

13. The non-transitory storage medium of claim 9, wherein the weighting factor is selected as a function of a magnitude of deviation between the reference model and the user-entered profile.

14. The non-transitory storage medium of claim 9, wherein modification of the open-loop delivery profile is constrained to prevent overshoot beyond the reference medicament delivery model.

15. A method for adapting medicament dosing parameters for a user, comprising:
determining, for a defined time segment, a deviation between a programmed basal medicament delivery amount used during open-loop operation and a basal medicament delivery amount applied during closed-loop operation;
computing a proportional adjustment factor based on the deviation;
applying the proportional adjustment factor to modify both: (i) a basal medicament delivery parameter associated with the time segment, and (ii) at least one bolus-related dosing parameter selected from an insulin-to-carbohydrate ratio and a correction factor; and
determining and delivering bolus medicament doses using the modified bolus-related dosing parameter while operating in the open-loop mode.

16. The method of claim 15, wherein the deviation is computed between a total basal medicament delivery amount during open-loop operation and a total basal medicament delivery amount during closed-loop operation for the same time segment.

17. The method of claim 15, wherein the proportional adjustment factor is derived as a ratio of closed-loop basal delivery to open-loop basal delivery.

18. The method of claim 15, wherein the proportional adjustment factor is constrained within predefined upper and lower bounds.

19. The method of claim 15, wherein applying the proportional adjustment factor to modify the basal medicament delivery parameter comprises scaling an hourly basal delivery rate.

20. The method of claim 15, wherein applying the proportional adjustment factor to modify the bolus-related dosing parameter comprises scaling at least one of:
an insulin-to-carbohydrate ratio, or
a correction factor.

21. The method of claim 15, wherein delivering bolus medicament doses uses the modified bolus-related dosing parameter during a subsequent open-loop bolus event.

22. The method of claim 15, wherein the proportional adjustment factor is derived from closed-loop basal delivery history averaged over a plurality of days.
